(19) United States
(12) Patent Application Publication
(10) Pub. No.: US 2012/0190905 A1
(43) Pub. Date: Jul. 26, 2012

(54) OLEFIN SELECTIVE MEMBRANE COMPRISING AN IONIC LIQUID AND A COMPLEXING AGENT

(75) Inventors: Johnathan T. Gorke, Saint Paul, MN (US); Shawn D. Feist, Midland, MI (US); Scott T. Matteucci, Midland, MI (US); Peter N. Nickias, Midland, MI (US)

(73) Assignee: DOW GLOBAL TECHNOLOGIES LLC, Midland, MI (US)

(21) Appl. No.: 13/384,840

(22) PCT Filed: Sep. 16, 2010
(86) PCT No.: PCT/US10/49139

§ 371 Date: Jan. 19, 2012

(30) Foreign Application Priority Data
Sep. 25, 2009 (US) 61245788

(51) Int. Cl. C07C 7/144; B01D 21/00

(52) U.S. Cl. 585/818; 422/527

(57) ABSTRACT

An improved ionic liquid membrane and its preparation for separation of olefins/paraffins is described. The membrane comprises an ionic liquid with a metal salt. The ionic liquid includes a choline salt, selected from choline, chloride/hydroxide/bitratrate, phosphatidylcholine and is a deep eutectic liquid. The metal salt selected from silver, copper, gold, mercury, cadmium, zinc with choloride, nitrate, tetrafluoroborate, triflate, cyanide, thiocyanide, tetraphenylborate as anion. The ionic liquid is eutectic or a so-called deep eutectic liquid. The experimental examples use choline chloride, urea and silver nitrate/chloride and are tested for methane/ethene separation.

### CROSS REFERENCE TO RELATED APPLICATIONS

This application is a non-provisional application claiming priority from the U.S. Provisional Patent Application No. 61/245,788, filed on Sep. 25, 2009, entitled “OLEFIN SELECTIVE MEMBRANE COMPRISING AN IONIC LIQUID AND A COMPLEXING AGENT,” the teachings of which are incorporated by reference herein, as if reproduced in full hereinbelow.

### BACKGROUND

1. Field of the Invention

The invention relates to the field of olefin selective membranes. More particularly, it relates to olefin selective membranes that include ionic liquids with low olefin sorption capacity to increase separation efficiency.

2. Background of the Art

The separation of olefins from mixtures with paraffins is an important process for producing many chemicals, including but not limited to polyethylene, polypropylene, and other polymers based on olefinic monomers. Unfortunately, olefin/paraffin separations are both capital and energy intensive.

Currently, cryogenic distillation is the dominant commercially employed method for separating olefins from mixtures with paraffins of the same carbon number, at volumes that are necessary for the polymer industry. Other separation techniques that have been tested and failed for this type of separation include ceramic membranes, polymer membranes, and pressure swing absorption. Unfortunately, ceramic membranes tend to be fragile and therefore cannot be readily made into modules that are sufficient for separations; polymer membranes are often unable to produce a product stream that is sufficiently pure to meet requirements for polymer grade feed stocks; and pressure swing absorption requires complex systems containing large amounts of media that are frequently inadequate to meet volume requirements.

Those in the industry have attempted to overcome the above drawbacks, particularly those relating to purity and/or production, by including in membranes certain metal ions which have the capacity to interact with pi-bonds. For example, one approach has been to employ an aqueous solution of such metal ions supported on polymeric membranes. However, these materials require water in order to enable facilitated transport to occur, which leads to unacceptably expensive steps to hydrate feed streams and subsequently dry the permeate streams.

Certain ionic liquids have been shown to improve olefin purity for higher hydrocarbons (i.e., pentene, hexene, and isoprene), which has alleviated the need for hydrating the feed stream and then drying the permeate stream. These liquids also eliminate the need to evaporate solvent, since the ionic liquids themselves have an inherently low vapor pressure. However, the ionic liquid that has been employed has required saturation with C5 and higher hydrocarbons. Where such hydrocarbons are not used, the result is an impermeable salt layer.

Another method has included using ionic liquids with complexing metal salts that have the ability to sorb higher concentrations of olefins than of paraffins. Although such high sorbing materials may present good pure gas selectivities, in mixed gas separations these membranes tend to plasticize because of the high concentration of olefin in the membrane. Such plasticization reduces the olefin/paraffin selectivity in mixed gas systems, which is detrimental to membrane performance. This loss of performance results from the competition between normal Fickian diffusion, which reduces membrane selectivity towards olefins and increases permeability for all penetrant gases during plasticization, and facilitated transport.

Thus, what is needed in the art is a means of enabling separation of olefins from mixtures with paraffins, which does not suffer from the drawbacks and problems recited hereinabove.

### SUMMARY OF THE INVENTION

In one aspect the invention is a membrane for separation of olefins from paraffins, comprising as a matrix an ionic liquid having an olefin sorption capability defined as having a Henry's Law Constant for ethylene that is greater than 130 bar (13000 kPa) at a selected membrane operation temperature, the matrix containing at least one metal salt capable of facilitating an olefin; the matrix being suitable such that, when the membrane is placed into contact with a mixture of olefins and paraffins at the selected membrane operation temperature, the olefins are substantially separated from the paraffins.

In another aspect the invention is a method of preparing a membrane for separating olefins from paraffins in a mixture thereof, comprising adding at least one metal salt capable of facilitating an olefin to an ionic liquid having an olefin sorption capability defined as having a Henry's Law Constant for ethylene that is greater than 130 bar (13000 kPa) at a selected membrane operation temperature, to form a membrane that, when in contact with a mixture of olefins and paraffins at the selected membrane operation temperature, is capable of substantially separating olefins from paraffins in a mixture thereof.

In yet another aspect the invention provides a method of separating olefins from paraffins contained together in a mixture, the method comprising contacting, at a selected membrane operation temperature, a feed stream, containing an olefin and a paraffin, with a membrane having as a matrix an ionic liquid having an olefin sorption capability defined as having a Henry's Law Constant for ethylene that is greater than 130 bar (13000 kPa) at the selected membrane operation temperature, the matrix containing at least one metal salt capable of facilitating an olefin, the matrix being suitable such that the olefin is substantially separated from the paraffin.

### DETAILED DESCRIPTION OF THE EMBODIMENTS

In general the invention is a membrane that offers the benefit of enabling highly selective facilitated transport of olefin molecules, and discouraging Fickian diffusion, thereby effecting excellent separation of mixtures. Because additional processing steps, such as hydration and/or evaporation are not required, and the membrane does not suffer from a reduction in olefin/paraffin selectivity due to plasticization, capital and energy costs are reduced. Furthermore, the membrane constituents are easily synthesized.

The membrane comprises at least one ionic liquid that contains at least one metal salt capable of facilitating an olefin. As defined herein, the phrase “capable of facilitating an olefin” means that the metal salt is able to interact with an olefin in such a way that it provides facilitated transport of the olefin across the membrane. In some embodiments a deep eutectic solvent may be used. As used herein, the term “ionic liquid” means a liquid ionic material, and “deep eutectic solvent” means a mixture of compounds (that may or may not be ionic in their pure state or liquid at ambient temperature) that forms a eutectic, i.e., an ionic solvent that displays a melting point that is different from that of any one of the compounds included in it. Thus, “deep eutectic solvents,” as the term is used herein, represents just one subgroup of “ionic liquids” and are included as possible selections for the ionic liquid.

The ionic liquid may be selected from any that poorly sorb olefins and paraffins. By “poorly sorb” or “poor sorption” is meant that the ionic liquid exhibits a Henry's Law Constant for ethylene (“H ethylene”) that is greater than 130 bar (13000 kPa) at membrane operation temperature. In general this means that, when the ionic liquid is in contact with an olefin-containing mixture at 200 pounds per square inch gauge (psig) (1379 kPa) and 30° C., the ionic liquid sorbs no more than the equivalent of about 2 psig (13.8 kPa) of the olefin. Measurement of the sorption capability is thus dependent upon the character of both the penetrant mixture and of the specified olefin itself, and is measured using a “parallel pressure reactor” at the temperature at which the membrane will be operating for a desired separation, i.e., the membrane operation temperature, which may vary from −100° C. to 400° C. in a wide variety of applications. The “parallel pressure reactor (PPR)” is actually a system of several reactors oriented in parallel and maintained at a constant pressure. Pressure curves obtained therefrom are indicative of the solubility of any given penetrant in a matrix. It is an advantage of the present invention that, by requiring an ionic liquid that poorly sorbs olefins, Fickian diffusion and plasticization of the membrane are reduced, and because Fickian diffusion is reduced, facilitated transport and, thus, selectivity, of the membrane are concomitantly enhanced and stabilized.

Application of Henry's Law may be carried out as follows. The Henry's Law Constant (“H”) for most gases (including ethylene) may be determined using the formula:

H=A.V^{B}

where A and B are constants, V is ionic liquid molar volume in L/mol, and H has units of bar. For ethylene A and B have values of 15.7 and −1.67 at 25° C., respectively. Those skilled in the art will easily be able to obtain the A and B constant values for other olefins from recognized reference sources. Solubility, S, in units of L/(bar mol) may then be determined as follows:

$$S = \frac{1}{PV ( \frac{H}{P} - 1 )}$$

where P is the feed pressure. It will be clear to those skilled in the art that a relatively high Henry's Law Constant implies a relatively low solubility of the given gas in the ionic liquid, i.e., a relatively low sorption of that gas in the ionic liquid.

Examples of suitable ionic liquids may include, generally, combinations of quaternary ammonium salts with hydrogen donors such as amines and carboxylic acids. These salts include the quaternary ammonium cations that characteristically retain their charge, regardless of pH, and are synthesized by complete alkylation of ammonia or other amines. In one non-limiting embodiment, a combination of choline chloride (2-hydroxy-N,N,N-trimethylammonium chloride, also referred to as hepacholine, bicolina or lipotril) and urea is selected. The choline chloride may be prepared by the industrial Davy process, using as starting materials ethylene oxide, hydrochloric acid, and trimethylamine. Those skilled in the art will recognize that the combination of choline chloride and urea, particularly in a 1:2 molar ratio, is eutectic, with a melting point as low as 12° C. In other non-limiting embodiments, other choline salts, such as choline hydroxide, choline bitartrate, phosphatidylcholine, and combinations thereof may be used. A few examples may be seen in Table 1 hereinbelow, which shows the Henry's Law Constant for ethylene (“H ethylene”) at 30° C. However, it is important to remember that the sorption capability, as defined by the H ethylene value, is determined for the membrane operation temperature, and therefore may differ significantly from the values shown for a membrane operation temperature of 30° C.

TABLE 1

| Cation | Anion | H Ethylene, bar |
|---|---|---|
| BMIM | PF6 | 195 |
| EMIM | Tf2N | 138 |
| EMIM | BF4 | 322 |
| EMIM | TfO | 227 |
| HMIM | Tf2N | 157 |
| MMIM | MeSO4 | 264 |
| ChCl | Gly | 136 |
| ChCl | EG | 175 |
| ChCl | U | 230 |

KEY:
BMIM is 1-butyl-3-methylimidazolium
EMIM is 1-ethyl-3-methylimidazolium
HMIM is 1-hexyl-3-methylimidazolium
MMIM is 1,3-dimethylimidazolium
ChCl is choline chloride
PF6 is hexafluorophosphate
Tf2N is bis(trifluoromethane)sulfonimide
BF4 is tetrafluoroborate
MeSO4 is methyl sulfate
U is urea
Gly is glycerol
EG is ethylene glycol
TfO is trifluoromethanesulfonate

Added to the ionic liquid in the present invention is any metal salt which contains a metal cation that is capable of facilitating an olefin, which implies that the metal salt is “pi-bondphilic.” Non-limiting examples of pi-bondphilic metal cations may be found in Groups X to XII (10 to 12) of the Periodic Table, and in certain particular embodiments, in Groups XI and XII (11 and 12) of the Periodic Table. One example of such a cation is silver cation (Ag⁺), and salts containing other cations, such as copper (Cu⁺), gold (Au⁺), zinc (Zn²⁺), mercury (Hg²⁺), cadmium (Cd²⁺), or a combination thereof, may also or alternatively be selected. In particular non-limiting embodiments, salts of copper or silver may be selected, and of these silver salts may be especially useful. Suitable anions for the salts may include, but are not limited to, chloride, nitrate, borofluoride, and combinations thereof. In certain non-limiting embodiments metal salts useful in the present invention may include silver chloride (AgCl), silver nitrate (AgNO₃), silver tetrafluoroborate (AgBF₄), silver triflate (AgCF₃SO₃), silver cyanide (AgCN), silver thiocyanide (AgSCN), silver tetraphenylborate (AgB(C₆H₅)₄), and combinations thereof. In general, the salts serve as facilitating agents, which means that they weakly bind and then release the penetrant. Because they tend to select pi-bonds with which they interact, they are therefore instrumental in separating the olefins from similar paraffins present in a penetrant mixture.

In proportion the metal salts may be included in the ionic liquid at a concentration ranging from 50 parts per million (ppm) to a point of saturation. Thus, actual maximum (saturation) concentration will depend upon the selection of matrix material and salt. In general it is preferred to use a relatively high concentration, since greater levels of salts tend to promote higher degrees of transport and thus, more selective separations and/or higher olefin flux.

The membrane matrix, containing the metal salt capable of facilitating an olefin, is incorporated into an appropriate housing or other vehicle, generally within an apparatus enabling flow of an appropriate feed stream. Such housing or other vehicle may variously be a column or cell, which may include a support made of a polymer, such as a cellulosic fiber or glass fiber, onto which a thin layer of the matrix has been applied. For example, a selective layer of ionic liquid that is from 20 μm to 10,000 μm in thickness may be used in some embodiments of the present invention. For example, glass or cellulosic fiber may be effectively supported on wax paper

The membranes of the present invention may find particular application for separation of olefins from paraffins particularly in commercial settings. Separation using the membranes may, in particular non-limiting embodiments, in at least substantial separation of the two types of hydrocarbons. By “substantial” or “substantially” herein is meant that there is a higher concentration (i.e., a higher mole percent) of olefin in the permeate stream than in the feed stream.

### EXAMPLES

### Example 1 and Comparative Example 1

### Synthesis of Choline Chloride-Urea Ionic Liquid (Example 1)

An amount of pure (99 weight percent) choline chloride is added to a flask. Also added to the flask is an amount of pure (99 weight percent) urea, such that the molar ratio of choline chloride to urea is 1:2. The mixture is stirred at 250-500 revolutions per minute (rpm) at 80° C. until a homogeneous liquid forms, after about one hour. To this ionic liquid is added and dissolved an amount of silver chloride (AgCl) to a point near to or at saturation. This composition is denoted hereafter as ChCl:U2 AgCl.

### Synthesis of 1-butyl-3-methylimidazolium bis(trifluoromethane)sulfonamide ionic liquid (Comparative Example 1)

An amount of the ionic liquid 1-butyl-3-methylimidazolium chloride (more than 95 weight percent) is added to a 1-neck round bottom flask on a stir plate. Deionized water is added to the ionic liquid (5:1 weight/weight (w/w)) and the ionic liquid is allowed to dissolve therein. An exchange metal salt, lithium bis(trifluoromethane)sulfonamide), is then added such that there is a 1:1 molar ratio of ionic liquid to exchange salt. The sides of the flask are washed down with deionized water, for 10:1 w/w total water-to-ionic liquid ratio. The ionic liquid containing the exchange salt is then stirred at 250-500 revolutions per minute (rpm) for at least 12 hours at ambient temperature. The remainder is then washed five (5) times with a 5:1 weight/weight (w/w) ratio of deionized water to starting ionic liquid. The remainder exhibits a single phase.

After the final decanting, a 1:1 volume ratio of ethanol, which is substantially free of water, is added to the ionic liquid. The flask is then attached to a rotary evaporator. The evaporator is run at 50° C. for at least two hours to remove the ethanol and any remaining water. The remaining content of the flask is 1-butyl-3-methylimidazolium bis(trifluoromethane)sulfonamide. This compositions is denoted hereafter as BMIM:Tf2N. No metal salt capable of facilitating an olefin is added.

### Preparation of the Example 1 and Comparative Example 1 Membranes

Between 0.5 and 2 g of the Example 1 ionic liquid (containing silver chloride as a metal salt capable of facilitating an olefin) is placed on a glass fiber sample supported by wax paper, and a similar amount of the Comparative Example 1 ionic liquid is placed on another glass fiber sample supported by wax paper. Each sample is loaded into a permeation cell, and each cell is fixed into a pure gas permeation system. The permeation system is a constant volume/variable pressure system that is conventionally used in the art. Both samples are exposed to a vacuum at least 16 hours at 70° C. prior to testing.

Testing of Matrix Olefin Sorption Properties

Samples (5 ml each) of each membrane matrix are placed in vials in a parallel pressure reactor (PPR). The samples are exposed to 200 psi (1379 kPa) ethylene at 30° C. Pressure of the ethylene is maintained by the PPR at 200 psi (1379 kPa) for the duration of each test. Uptake of each sample is determined from the difference of the integrated area under the curve at constant pressure of 200 psi (1379 kPa) and the sample pressure curve.

### Testing of the Membranes' Gas Transport Properties

The Example 1 and Comparative Example 1 membranes are each first exposed to methane at 15 pounds per square inch gauge (psig) (103.4 kPa) until the rate of pressure increase reaches a steady state (i.e., less than a 0.5 percent change in pressure increase over a period of at least 10 minutes). Subsequently, methane feed pressure is raised to 45 psig (310.3 kPa). Once methane reaches a steady state in a system containing a particular membrane, that system is evacuated for at least two (2) hours, but typically for at least sixteen (16) hours. Ethylene permeation tests are conducted in a manner similar to the methane tests. Methane permeability experiments are then repeated at 15 psig (103.4 kPa) to determine if plasticization has occurred.

### Effecting a Separation

A feed comprising 50 mole percent ethylene and 50 mole percent methane is prepared and contacted with each membrane under a pressure differential across the membrane of 8 bar (800 kPa). Once a given system has reached steady state operation, samples are taken of both the permeate stream and the retentate stream. For the Example 1 membrane, the permeate stream contains at least 75 mole percent of ethylene, and the retentate stream contains at least 80 mole percent of methane. For the Comparative Example 1 membrane, the permeate and retentate streams each contain 50 mole percent of ethylene and 50 mole percent of methane.

### Examples 2-3 and Comparative Examples 2-7

Using the Example 1 and Comparative Example 1 methods, respectively, two additional example compositions (Examples 2-3) and six comparative compositions (Comparative Examples 2-7) are prepared as membranes, with the compositions shown in Table 2.

TABLE 2

| | | Ethylene Sorption at | |
|---|---|---|---|
| | | 1800 seconds | |
| Sample | Sample | at 30° C. in PPR, | |
| Identification | Composition | psig (kPa) | |
| Comparative | ChCl:U2* | 1.5 | (10.3) |
| Example 2 | | | |
| Comparative | ChCl:Gly2* | 1.45 | (10) |
| Example 3 | | | |
| Comparative | ChCl:EG2* | 1.95 | (13.4) |
| Example 4 | | | |
| Comparative | BMIM[AOT] | 2.8 | (19.3) |
| Example 5 | | | |
| Comparative | BMIM[Tf2N] | 8.0 | (55.2) |
| Example 6 | | | |
| Comparative | BMIM[BF4] | 7.4 | (51.0) |
| Example 7 | | | |
| Example 2 | ChCl:U2 AgCl | 0.5 | (3.4) |

| | | |
|---|---|---|
| Example 3 | ChCl:U2 AgNO₃ | — |

Key:
ChCl is choline chloride
U is urea
Gly is glycerol
EG is ethylene glycol
2* refers to stoichiometry, i.e., 2 moles of urea, glycerol, or EG per mole of choline chloride.
BMIM[AOT] is 1-butyl-3-methylimidazolium dioctylsulfosuccinate
BMIM[Tf2N] is 1-butyl-3-methylimidazolium bis(trifluoromethane)sulfonimide
BMIM[BF4] is 1-butyl-3-methylimidazolium tetrafluoroborate

It is noted that, while Comparative Examples 2-4 meet the inventive ionic liquid sorption requirement for membrane operation at 30° C., they lack a metal salt capable of facilitating an olefin, while the ionic liquids employed in Comparative Examples 5-7 have Henry's Law Constants for ethylene that are below 130 bar (13000 kPa) at the same membrane operation temperature. From the data under the heading “Ethylene Sorption” it may be inferred that the membrane of Example 2, having an extremely low ethylene sorption, will as a result experience a significant reduction in plasticization, which translates to a significant decrease in ethylene/methane selectivity. Data is not available for ethylene sorption for Example 3, but a similarly low ethylene sorption and reduction in plasticization is anticipated.

### Examples 4-5 and Comparative Example 8

Membranes representing two additional examples of the invention (Examples 4 and 5) and one comparative example (Comparative Example 8) are tested for pure gas ethylene/methane selectivity using the method described in the section titled “Testing of the Membranes' Gas Transport Properties” hereinabove. Results are shown in Table 3. These data show the increased ethylene/methane selectivity in a low ethylene sorbing ionic liquid filled with two different silver salts, silver chloride (AgCl) and silver nitrate (AgNO₃). It should be noted that the ionic liquid used in Comparative Example 8 exhibits a reversal in selectivity behavior when compared with the same ionic liquid filled with a silver salt, as can be seen in Examples 4 and 5, i.e., the unfilled membrane is methane selective, whereas the silver salt filled membrane is ethylene selective.

TABLE 3

| Sample Identi- fication | Matrix | Metal Salt | $\frac{{C}_{2} {H}_{4}}{{CH}_{4}} Selectivity$ | Feed pressure, psig (kPa) |
|---|---|---|---|---|
| Comparative | Choline chloride + | None | 0.7 | 45 |
| Example 8 | Urea | | | (310.3) |
| Example 4 | Choline chloride + | AgCl | 1.5 | 40 |
| | Urea | | | (275.8) |
| Example 5 | Choline chloride + | AgNO₃ | 5.1 | 15 |
| | Urea | | | (103.4) |
| | | | 3.8 | 45 |
| | | | | (310.3) |

What is claimed is:

1. A membrane for separating olefins from paraffins, comprising as a matrix an ionic liquid having an olefin sorption capability defined as having a Henry's Law Constant for ethylene that is greater than 130 bar (13000 kPa) at a selected membrane operation temperature, the matrix containing at least one metal salt capable of facilitating an olefin; the matrix being suitable such that, when the membrane is placed into contact with a mixture of olefins and paraffins at the selected membrane operation temperature, the olefins are substantially separated from the paraffins.

2. The membrane of claim 1 wherein the ionic liquid includes a choline salt selected from the group consisting of choline chloride, choline hydroxide, choline bitartrate, phosphatidylcholine, and combinations thereof.

3. The membrane of claim 1 wherein the ionic liquid is a deep eutectic solvent.

4. The membrane of claim 1 wherein the metal salt capable of facilitating an olefin contains a pi-bondphilic cation selected from the group consisting of Ag⁺, Cu⁺, Au⁺, Hg²⁺, Cd²⁺, Zn²⁺, and combinations thereof.

5. The membrane of claim 1 wherein the metal salt capable of facilitating an olefin contains an anion selected from the group consisting of chloride, nitrate, tetrafluoroborate, triflate, cyanide, thiocyanide, and tetraphenylborate.

6. The membrane of claim 7 wherein the metal salt capable of facilitating an olefin is silver chloride, silver nitrate or a combination thereof.

7. A method of preparing a membrane for separating olefins from paraffins in a mixture thereof, comprising adding at least one metal salt capable of facilitating an olefin to an ionic liquid having an olefin sorption capability defined as having a Henry's Law Constant for ethylene that is greater than 130 bar (13000 kPa) at a selected membrane operation temperature, to form a membrane that, when in contact with a mixture of olefins and paraffins at the selected membrane operation temperature, is capable of substantially separating the olefins from the paraffins.

8. A method of separating olefins from paraffins contained together in a mixture, the method comprising contacting, at a selected membrane operation temperature, a feedstream, containing an olefin and a paraffin, with a membrane having as a matrix an ionic liquid having an olefin sorption capability defined as having a Henry's Law Constant for ethylene that is greater than 130 bar (13000 kPa) at the selected membrane operation temperature, the matrix containing at least one metal salt capable of facilitating an olefin, the matrix being suitable such that the olefin is substantially separated from the paraffin.
